(19) United States
(12) Patent Application Publication
(10) Pub. No.: US 2018/0296463 A1
(43) Pub. Date: Oct. 18, 2018

(54) PRE-MIX AND PROCESS FOR PREPARING PERSONAL CARE COMPOSITIONS, COMPOSITION PROMOTING IMPROVED AND LONG-LASTING CLEANSING SENSORY EXPERIENCE, IMPROVED ORAL CARE COMPOSITION

(71) Applicant: Sanofi, Bridgewater, NJ (US)

(72) Inventors: Honorio Obias, Ooltewah, TN (US); Daniel Hekman, Chattanooga, TN (US); Shane Smith, Ooltewah, TN (US); Thomas Knopp, Signal Mountain, TN (US); Sherry McGraw, Bridgewater, NJ (US); Charisse Winecki, Chattanooga, TN (US)

(21) Appl. No.: 15/890,454

(22) Filed: Feb. 7, 2018

Related U.S. Application Data
(63) Continuation of application No. 14/132,879, filed on Dec. 18, 2013, now abandoned.

(51) Int. Cl. A61K 8/97; A61Q 11/00; A61K 8/86; A61K 8/55; A61K 8/37; A61K 8/36; A61K 8/34; A61K 8/49

(52) U.S. Cl. CPC A61K 8/97; A61Q 11/00; A61K 8/86; A61K 8/55; A61K 8/4926; A61K 8/37; A61K 8/361; A61K 8/34

(57) ABSTRACT

The present invention provides a personal care cleansing composition, and also provides a pre-mix and a process for preparing personal care compositions that can be presented in liquid, semi-solid or solid forms. The composition of the invention comprises a unique combination of cleansing and soothing agents, and produces an improved and long-lasting cleansing sensory experience. In a an embodiment aimed for oral care, there is provided an alcohol-free mouthwash composition particularly useful for cavity prevention and treatment as well as white spots prevention for a subject wearing braces.

### FIELD OF THE INVENTION

The present invention is related to personal care preparations. Specifically, the present invention provides a personal care cleansing composition, and also provides a pre-mix and a process for preparing personal care compositions that can be presented in liquid, semi-solid or solid forms. The composition of the invention comprises a unique combination of cleansing and soothing agents, and produces an improved and long-lasting cleansing sensory experience. In an embodiment aimed for oral care, there is provided an alcohol-free mouthwash composition particularly useful for cavity prevention and treatment as well as white spots prevention for a subject wearing braces.

### BACKGROUND OF THE INVENTION

Personal care cleansing compositions are very important for human hygiene. Multiple factors affect their performance in the human body, including efficiency in cleansing, compatibility with different tissues, the feel produced to the user and suitability to different age, gender, preference profiles—to name just a few.

Some particular regions of the body present challenges for an effective cleansing. Body regions which are not flat or easily exposed can be considered as regions of difficult access, and tend to accumulate materials. Without proper cleansing, bacteria and other unwanted microorganisms generally thrive in these regions. Therefore, the development of personal care cleansing compositions suited for these challenges has been an important target to health care professionals.

The mouth, for instance, is a body region of particular attention in health care. Failure to provide an effective cleansing of the mouth leads to multiple problems, such as the formation of dental plaque—which is the primary source of dental caries, gingival and periodontal disease and tooth loss. Dental plaque is a mixed matrix of bacteria, epithelial cells, leukocytes, macrophages and other oral exudates. Bacteria comprise approximately three-quarters of the plaque matrix. Any given sample of dental plaque could contain as many as 400 different varieties of microorganisms. This mix includes both aerobic and anaerobic bacteria, fungi and protozoa. Even viruses have been found in samples of dental plaque. This matrix of organisms and oral exudates continues expanding and coalesces with other plaque growths situated nearby. The bacteria synthesize levans and glucans from sucrose found in the oral cavity providing energy for the microorganisms. These glucans, levans and microorganisms form an adhesive skeleton for the continued proliferation of plaque. The failure to retard or stop the proliferation of plaque is detrimental to oral health. Plaque formation leads to dental caries, gingival inflammation, periodontal disease and ultimately tooth loss.

Mouthwashes, oral rinses and other oral care compositions have been in use for many years. They are utilized by hospitals and other health care facilities and, in general, by consumers. Most commercially available mouthwashes and oral rinses contain a fairly high percentage of ethyl alcohol, as ethyl alcohol is well known for its antibacterial properties and said property is very desirable in any kind of composition that aims to promote an effective clean while delivering a cleansing sensation that lasts for the longest time possible. While these alcohol-containing compositions are generally safe and effective, there are several important reasons why an alcohol-free composition is desirable. Some of these reasons include: a) high alcohol oral compositions have been linked in some cases to increased incidence of mouth and throat cancer; b) a large number of persons cannot tolerate alcohol and must, for health reasons, avoid its use; c) alcohol-containing oral compositions can be a poison hazard to a small child; d) alcohol-containing compositions are often abused by alcoholics; e) alcohol-containing oral compositions must be avoided by some persons because of religious convictions; and f) alcohol in a composition may irritate the protective layers of the mouth, throat and skin, or dry out inflamed tissues.

Therefore, while there are enough reasons to eliminate alcohol from these kinds of compositions, its antibacterial performance and cleansing experience should be replaced by other ingredients so that the compositions present the same efficiency and cleansing experience to users. Prior art comprises several examples of such approaches.

Document WO 94/16674 presents an antiseptic mouthwash composition with reduced alcohol levels. The inventors attribute the efficacy of said composition to the combination of four known active ingredients, i.e. thymol, eucalyptol, menthol and methyl salicylate, plus the addition of propylene glycol and glycerin as co-solvents.

U.S. Pat. No. 5,145,664 discloses an alcohol-free mouthwash composition containing as main ingredients sodium chloride, sodium bicarbonate, polysorbate 20, sodium saccharin, methylparaben and propylparaben, peppermint oil and, as an antibacterial agent, benzalkonium chloride.

U.S. Pat. No. 5,431,903 discloses an oral composition providing improved oral cleansing properties by the use of a mixture comprising one surfactant, such as sodium lauroyl sarcosinate or polysorbate 20, an enzyme, e.g. endoglycosidade or papain, a chelating agent, a fluoride ion source and a suitable carrier.

Document WO 95/06455 discloses toothpaste and tooth gel compositions containing baking soda and an alkali metal pyrophosphate salt. The compositions described therein comprise as main active ingredients: sodium bicarbonate, tetrasodium pyrophosphate, sodium fluoride, sorbitol, glycerin, sodium saccharin, sodium lauryl sulfate, sodium lauroyl sarcosinate, thickening silica and hydrated silica.

The previously cited documents show a trend of mixing surfactants and other cleansing agents as an alternative to the use of ethyl alcohol as antibacterial agent.

U.S. Patent document 2009/035229 moves away from that strategy by disclosing an aqueous based composition for oral hygiene treatment comprising a tetracycline-based antibiotic as the antibacterial agent of choice.

U.S. Patent application number 2009/017101 is related to a solid composition containing a chelating agent, a fatty acid salt (ricinoleic acid salt, such as sodium ricinoleate), titanium or silicon dioxide and an abrasive agent.

Finally, document WO 2012/021415 discloses a zinc-based oral composition. Zinc chloride and hydrogen peroxide are referred to as the antibacterial agents of choice in said composition, which also comprise cetylpyridinium chloride as cleansing agent.

To the best knowledge of the inventors, the problem of providing a personal care cleansing composition promoting an efficient deep clean while rendering a cleansing sensory experience that lasts for the longest time possible, has not been adequately solved so far. In this context, the development or design of new combinations of ingredients to provide an improved and long-lasting cleansing sensory experience is highly desirable and would increase the effectiveness of the formulation. This would also reduce the number of applications of the product through the day, thus, being extremely beneficial to the patients/users. The present invention solves these and other technical problems and further provides a personal care formulation comprising a unique and not previously explored combination of cleansing and/or soothing agents to achieve an improved and long-lasting refreshing and deep clean effect.

### SUMMARY OF THE INVENTION

The present invention provides: a personal care cleansing composition that produces a long lasting cleansing sensory experience; a pre-mix for preparing personal care compositions; a process for preparing personal care compositions; an improved oral care composition suited for cavity prevention/treatment and white spots prevention especially suited for a subject wearing dental braces.

The inventive concept underlying these objects of the invention is the use of a unique and not previously explored combination of cleansing and/or soothing agents to achieve an improved and long-lasting refreshing and deep clean effect. The composition of the invention comprises a well-balanced mixture of multi-charged, negatively-charged, positively-charged and uncharged cleansing and/or soothing agents. Preferably said composition comprises seven cleansing and/or soothing agents. In an embodiment, the composition of the invention is for oral care, is alcohol free and comprises a mixture of flavor-producing soothing agents, optionally further comprising sodium fluoride.

It is therefore a first object of the invention a liquid, semi-solid or solid personal care cleansing composition that produces an improved and long-lasting cleansing sensory experience, said preparation comprising a unique combination of cleansing and soothing agents. In an embodiment, said composition comprises multi-charged, negatively-charged, positively-charged and uncharged cleansing and/or soothing agents. Optionally, the composition of the invention may also comprise other ingredients selected from: moisturizer, solvent, humectant, sweetener, preservative, buffer, metal chelator, color additive and combinations thereof. When for oral care, the composition of the invention comprises flavor-producing soothing agents such as botanical, tingling, mint and sweet flavor. In another embodiment, the composition of the invention comprises agents selected from mild cleansing agents, non-ionic surfactant cleansers, quaternary surfactant cleansers, multiquaternary surfactants, phospholipids, alkyl polyphosphates, alkyl betaines, alkyl hydroxysultaines, alkyl sulfosuccinates, polyphosphates, antiseptics, mild amino acid surfactant cleansers, cooling agents, soothing mint flavor agents, soothing tingling sensate, sweet soothing agents, soothing botanical agents and soothing flavor agents. In an embodiment, the personal care composition of the invention is alcohol free. In another embodiment, the personal care composition of the invention further comprises 0.01 to 0.5% of sodium fluoride for preventing cavities and the annoying white spots formation.

It is an additional object of the present invention a pre-mix for preparing personal care cleansing compositions. The pre-mix of the invention comprises a combination of multi-charged, negatively-charged, positively-charged and uncharged cleansing and/or soothing agents and is used for preparing liquid, semi-solid or solid personal care cleansing compositions as described above. The pre-mix of the invention is particularly useful for subsequent introduction of carriers or inert ingredients, therefore providing an economically attractive solution for producing multiple different compositions, either in different forms or for different uses. The pre-mix of the invention also provides logistic advantages for mass production.

It is an additional object of the present invention a process for preparing personal care compositions, comprising the following steps:
adding to a container, individually or pre-mixed, multi-charged, negatively-charged, positively-charged and uncharged cleansing and/or soothing agents; and
completing the volume with the acceptable carrier for liquid, semi-solid or solid final form.

Optionally, the process may also comprise the introduction, either individually or pre-mixed, of other ingredients selected from: moisturizer, solvent, humectant, sweetener, preservative, buffer, metal chelator, color additive and combinations thereof.

In an embodiment, the process for preparing personal care compositions comprises the following steps:
adding to a container, individually or pre-mixed, at least one uncharged cleansing agent, at least one positively-charged cleansing agent, at least one multi-charged cleansing agent, at least one negatively-charged cleansing agent and then homogenizing the resulting mixture;
adding to the container comprising the previously formed mixture, individually or pre-mixed, at least one soothing tingling sensate agent, at least one soothing mint flavor agent, at least one soothing botanical agent and at least one soothing flavor agent; and
completing the volume with deionized, sterile or purified water.

These and other objects of the invention will become more readily appreciated by those skilled in the art upon the detailed description below.

### BRIEF DESCRIPTION OF THE FIGURES

FIG. 1 shows a graphic representation of the assessment of the four attributes measured (color, taste, clean, and overall) of the invention indicated with capital letters. Mean scores are represented as follows: 0=poor, 10=excellent.

FIG. 2 shows a graphic representation of the assessment of the cleansing-related sensory experience of some embodiments of the invention indicated with capital letters. Clean mean scores are represented as follows: 0=poor; 10=excellent.

FIG. 3 shows a graphic representation of the assessment of the color-related sensory experience of some embodiments of the invention indicated with capital letters. Color mean scores are represented as follows: 0=poor;

10=excellent.

FIG. 4 shows a graphic representation of the assessment of the Taste-related sensory experience of some embodiments of the invention indicated with capital letters. Taste mean scores are represented as follows: 0=poor; 10=excellent.

FIG. 5 shows a graphic representation of the assessment of the Overall cleansing-related sensory experience of some embodiments of the invention indicated with capital letters. Overall clean mean scores are represented as follows: 0=poor; 10=excellent. FIG. 6 shows a graphic representation of the assessment of the combined sensory experience (Color (20%)+Taste (20%)+Clean (20%)+Overall (40%)) of some embodiments of the invention indicated with capital letters. Scores are represented as follows: 0=poor; 10=excellent.

### DETAILED DESCRIPTION OF THE INVENTION

The inventors aimed to solve the problems related to the preparation of a composition that produces a cleansing sensory experience in personal care compositions. The inventive concept underlying the invention is the use of a unique and not previously explored combination of cleansing and/or soothing agents to achieve an improved and long-lasting refreshing and deep clean effect.

Previously, both the inventors and others attempted to solve this problem by multiple approaches, but the traditional and known personal care cleansing compositions used so far have yet to deliver an effective and absolute answer to these problems. The inventors solved this technical problem by using a combination of agents constituted by multi-charged, negatively-charged, positively-charged and uncharged cleansing and/or soothing agents, which can be used in the form of pre-mix or in the form of a ready-to-use composition. The pre-mix of the invention is used for preparing liquid, semi-solid or solid personal care cleansing compositions. The pre-mix of the invention is particularly useful for subsequent introduction of carriers or inert ingredients, therefore providing an economically attractive solution for producing multiple different compositions, either in different forms or for different uses. The pre-mix of the invention also provides logistic advantages for mass production.

The present invention therefore provides: a personal care cleansing composition that produces a long lasting cleansing sensory experience; a pre-mix for preparing personal care compositions; a process for preparing personal care compositions; an improved oral care composition suited for cavity prevention/treatment and white spots prevention especially suited for a subject wearing braces.

The composition of the invention preferably comprises seven cleansing and/or soothing agents. In an embodiment, said composition comprises four cleansing agents and three soothing agents. In another embodiment, the composition of the invention is for oral care, is alcohol free and comprises a mixture of flavor-producing soothing agents, optionally further comprising sodium fluoride. By “alcohol free” it is hereby intended to mean ethanol free, thus avoiding ethanol abuse when using such oral care composition. The composition of the invention may also comprise flavor-producing soothing agents such as botanical, tingling, mint and sweet flavor. In an embodiment, said combination is constituted by agents selected from mild cleansing agents, non-ionic surfactant cleansers, quaternary surfactant cleansers, multiquaternary surfactants, phospholipids, alkyl polyphosphates, alkyl betaines, alkyl hydroxysultaines, alkyl sulfosuccinates, polyphosphates, antiseptics, mild amino acid surfactant cleansers, cooling agents, soothing mint flavor agents, soothing tingling sensate, sweet soothing agents, soothing botanical agents and soothing flavor agents. A composition of the present invention may comprise anyone of the following cleansing and/or soothing agents: poloxamer 407, chlorhexidine, octenidine, lecithin, cephalin, sphingomyelin, phosphatidylserine, phosphatidic acid, phosphatidylinositol, phosphatidylinositol phosphate, phosphatidylinositol diphosphate, phosphatidylinositol triphosphate, nucleosidic polyphosphates (adenosine diphosphate, adenosine triphosphate, uridine triphosphate, thymidine triphosphate, cytidine triphosphate, guanosine triphosphate), glycosyl polyphosphates (inositol polyphosphate, glucose polyphosphate), sodium hydroxypropylphosphate laurylglucoside crosspolymer, phosphatidylinositol diphosphate, phosphatidylinositol triphosphate, cocamidopropyl betaine, behenyl betaine, cetyl betaine, decyl betaine, oleyl betaine, stearyl betaine, lauryl betaine, myristyl betaine, tallow betaine, cocamidopropyl hydroxysultaine, lauryl hydroxysultaine, erucamidopropyl hydroxysultaine, lauramido hydroxysultaine, oleamidopropyl hydroxysultaine, tallowamidopropyl hydroxyl sultaine, dioctyl sodium sulfosuccinate, disodium laureth sulfosuccinate, disodium lauryl sulfosuccinate, sodium hexametaphosphate, sodium trimetaphosphate, cetylpyridinium chloride, polysorbate 20, sodium lauroyl sarcosinate, tetrasodium pyrophosphate, benzyl alcohol, Chattem sensate blend 709053, a sugar free sweetener, Aloe vera, methyl salicylate, menthol and Chattem sensate blend 112683.

In an embodiment, the personal care composition of the invention comprises:
from 0.01 to 10% w/w of at least one uncharged cleansing agent;
from 0.01 to 10% w/w of at least one positively-charged cleansing agent;
from 0.001 to 10% w/w of at least one multi-charged cleansing agent;
from 0.001 to 10% w/w of at least one negatively-charged cleansing agent;
from 0.001 to 10% w/w of at least one soothing tingling sensate agent;
from 0.01 to 0.5% w/w of at least one soothing mint flavor agent;
from 0.01 to 1% w/w of at least one sweet soothing agent;
from 0.001 to 1% w/w of at least one soothing botanical agent;
from 0.001 to 5% w/w of at least one soothing flavor agent; and
from 0.1 to 99% w/w of water.

In another embodiment, the personal care composition of the invention comprises 0.01 to 0.5% of sodium fluoride in addition to the combination of cleansing and/or soothing agents. This composition prevents cavities and prevents the formation of the annoying white spots.

### Process for Preparing Personal Care Cleansing Compositions

The process for preparing personal care cleansing compositions of the invention comprises the following steps:
adding to a container, individually or pre-mixed, multi-charged, negatively-charged, positively-charged and uncharged cleansing and/or soothing agents; and
completing the volume with the acceptable carrier for liquid, semi-solid or solid final form.

Optionally, the process may also comprise the introduction, either individually or pre-mixed, of other ingredients selected from: moisturizer, solvent, humectant, sweetener, preservative, buffer, metal chelator, color additive and combinations thereof.

In an embodiment, there is provided a process for preparing personal care compositions comprising the following steps:
adding, individually or pre-mixed to a container comprising water, at least one uncharged cleansing agents, at least one positively-charged cleansing agent, at least one multi-charged cleansing agent, at least one negatively-charged cleansing agent and then homogenizing the resulting mixture;
adding, individually or pre-mixed to a container comprising the previously formed mixture, at least one soothing tingling sensate agent, at least one soothing mint flavor agent, at least one soothing botanical agent and at least one soothing flavor agent; and
completing the volume with deionized, sterile or purified water.

In a further embodiment, the process for preparing personal care compositions comprises the following steps:
adding, individually or pre-mixed to a container comprising water, from 0.01 to 10% w/w of at least one uncharged cleansing agent, from 0.01 to 10% w/w of at least one positively-charged cleansing agent, from 0.001 to 10% w/w of at least one multi-charged cleansing agent, from 0.001 to 10% w/w of at least one negatively-charged cleansing agent, and then homogenizing the resulting mixture;
adding, individually or pre-mixed to a container comprising the previously formed mixture, from 0.001 to 10% w/w of at least one soothing tingling sensate agent, from 0.01 to 0.5% w/w of at least one soothing mint flavor agent, from 0.01 to 1% w/w of at least one sweet soothing agent, from 0.001 to 1% w/w of at least one soothing botanical agent, from 0.001 to 5% w/w of at least one soothing flavor agent; and
completing the volume with deionized, sterile or purified water.

An alcohol-free personal care composition prepared as per the process above has improved and long-lasting refreshing and deep clean effect. This composition is particularly useful for oral care and may optionally comprise 0.01 to 0.5% of sodium fluoride in addition to the aforementioned combination of cleansing and/or soothing agents, and thus prevents cavities and annoying white spots from forming. This embodiment of alcohol-free personal care composition of the invention is therefore useful for the protection and maintenance of oral health.

The examples shown here are intended only to illustrate some of the many ways to carry out the invention, and should not, however, limit the scope of the same.

### EXAMPLE 1

### Improved Oral Care Composition

In an embodiment, there is provided an oral care composition comprising:
from 0.01 to 0.5% w/w of sodium fluoride;
from 0.5 to 5% w/w of poloxamer 407;
from 0.01 to 1% w/w of cetylpyridinium chloride;
from 0.01 to 10% w/w of polysorbate 20;
from 0.001 to 10% w/w of sodium lauroyl sarcosinate;
from 0.001 to 1% w/w of tetrasodium pyrophosphate;
from 0.1 to 5% w/w of benzyl alcohol;
from 0.01 to 0.5% w/w of flavoring agent;
from 0.01 to 1% w/w of a sugar free sweetener;
from 0.001 to 1% w/w of Aloe vera;
from 0.001 to 5% w/w of methyl salicylate;
from 0.01 to 2% w/w of menthol;
from 0.001 to 10% w/w of Chattem sensate blend 112683; and
from 0.1 to 99% w/w of water.

Sugar free sweeteners are preferably selected from sucralose, aspartame, neotame, stevia, sodium saccharin etc. Optionally, other ingredients may be present in the composition in order to achieve an incremental cleansing effect. Examples of acceptable ingredients include: moisturizer, solvent, humectant, preservative, buffer, metal chelator, color additive and combinations thereof.

### EXAMPLE 2

An example of ethanol free composition according to the invention is shown in table 1 below. This composition avoids ethanol abuse by adults and/or inadvertent ethanol exposure to children using such oral care composition. This composition is a mouthwash that contains sodium fluoride to prevent cavities and annoying white spots from forming.

TABLE 1
Oral care composition according to the invention.

| Ingredient | % w/w |
|---|---|
| Sodium Fluoride | 0.02 |
| Sorbitol | 10 |
| Xylitol | 2 |
| Poloxamer 407 | 2 |
| Propylene Glycol | 1.5 |
| Potassium Sorbate | 0.5 |
| Benzyl Alcohol | 0.8 |
| Chattem sensate blend 709053 | 0.1 |
| Cetylpyridinium Chloride | 0.1 |
| Sugar free sweetener | 0.1 |
| Polysorbate 20 | 0.1 |
| Aloe vera | 0.05 |
| Methyl Salicylate | 0.5 |
| Menthol | 0.2 |
| Sodium Lauroyl Sarcosinate | 0.1 |
| Chattem sensate blend 112683 | 0.08 |
| EDTA Calcium Disodium | 0.02 |
| Sodium Phosphates | 0.05 |
| Sodium Benzoate | 0.5 |
| Water | q.s.p. |

The composition as shown above not only provides cleansing, but also provides the feel of cleaning in and around dental braces, where brushing and flossing are difficult. Therefore, the composition is useful for the treatment of cavity and white spots prevention and provides improved and long-lasting refreshing and deep clean effect, thus providing maintenance of oral health.

### EXAMPLE 3

Some of the many possible embodiments of the invention were evaluated in order to investigate the most effective combinations of cleansing and soothing agents able to deliver an improved and long-lasting cleansing sensory experience. For prompt reference, table 2 discloses further details of compositions D and M, which were tested for several effectiveness factors.

TABLE 2
Oral care compositions D and M.

| | Comp. D | Comp. M |
|---|---|---|
| Ingredient | (% w/w) | (% w/w) |
| Sodium Fluoride | 0.02 | 0.05 |
| Sorbitol | 8 | 8 |
| Poloxamer 407 | 1 | 1 |
| Propylene Glycol | 1 | 1 |
| Potassium Sorbate | 0.6 | 0.2 |
| Benzyl Alcohol | 0.1 | 0.2 |
| Flavor Chattem sensate blend 709053 | 0.1 | 0.02 |
| Cetylpyridinium Chloride | 0.04 | 0.03 |
| Sugar free sweetener | 0.1 | 0.07 |
| Polysorbate 20 | 0.1 | 0.1 |
| Aloe vera | 0.05 | 0.05 |
| Menthol | 0.2 | 0.2 |
| Chattem sensate blend 112683 | 0.01 | 0.01 |
| EDTA Calcium Disodium | 0.001 | 0.001 |
| Sodium Phosphates | 0.12 | 0.12 |
| Sodium Benzoate | 0.5 | 0.3 |
| Water | q.s.p. | q.s.p. |

Multiple tests were performed with said compositions, as well with others having concentration ranges claimed herein and described above. The results are summarized in table 3 below. Products were offered in a monadic sequential format. Randomized presentation order was applied. The amount of composition used was 10 ml swished for 60 seconds and one time use for evaluation. Evaluations were timed a minimum of 2 hours apart to minimize fatigue. Compositions were evaluated using a 10 point quality rating scale (0=Poor—10=Excellent). The scores of all attributes for each product were averaged and weighted to determine a final product score. Each of the three individual attributes was weighted at 20% with the overall score weighted at 40%. All of the weighted scores were added together for the final product score.

TABLE 3
Cleansing effectiveness and other sensorial assessments
of some embodiments of the invention.

| | Color | Taste | Clean | Overall | Color | Taste | Clean | Overall | Final |
|---|---|---|---|---|---|---|---|---|---|
| Composition | (mean) | (mean) | (mean) | (mean) | (20%) | (20%) | (20%) | (40%) | Score |
| A | 7.5 | 6.3 | 6.5 | 6.4 | 1.5 | 1.26 | 1.3 | 2.56 | 6.62 |
| B | 7.3 | 6.3 | 5.5 | 6.3 | 1.46 | 1.26 | 1.1 | 2.52 | 6.34 |
| C | 6.8 | 5.5 | 6.3 | 5.8 | 1.36 | 1.1 | 1.26 | 2.32 | 6.04 |
| D | 7.2 | 7.8 | 7.2 | 7.3 | 1.44 | 1.56 | 1.44 | 2.92 | 7.36 |
| E | 6.3 | 6.2 | 5.5 | 6.2 | 1.26 | 1.24 | 1.1 | 2.48 | 6.08 |
| F | 3.2 | 5.3 | 3.5 | 4 | 0.64 | 1.06 | 0.7 | 1.6 | 4 |
| G | 7.3 | 7 | 6.7 | 7 | 1.46 | 1.4 | 1.34 | 2.8 | 7 |
| H | 7 | 7 | 6.8 | 6.7 | 1.4 | 1.4 | 1.36 | 2.68 | 6.84 |
| I | 7.3 | 5.7 | 6.2 | 5.8 | 1.46 | 1.14 | 1.24 | 2.32 | 6.16 |
| J | 7.5 | 5 | 5.5 | 5.4 | 1.5 | 1 | 1.1 | 2.16 | 5.76 |
| K | 3.5 | 5.3 | 5 | 5.1 | 0.7 | 1.06 | 1 | 2.04 | 4.8 |
| L | 7.2 | 6 | 6.2 | 6 | 1.44 | 1.2 | 1.24 | 2.4 | 6.28 |
| M | 6.8 | 6.6 | 5.3 | 6.2 | 1.36 | 1.32 | 1.06 | 2.48 | 6.22 |
| N | 5.5 | 6.3 | 6.2 | 6.2 | 1.1 | 1.26 | 1.24 | 2.48 | 6.08 |
| O | 5.3 | 6.8 | 5.5 | 6 | 1.06 | 1.36 | 1.1 | 2.4 | 5.92 |
| P | 6.7 | 6.5 | 5.8 | 6.5 | 1.34 | 1.3 | 1.16 | 2.6 | 6.4 |
| Q | 7.7 | 6.8 | 6.2 | 6.7 | 1.54 | 1.36 | 1.24 | 2.68 | 6.82 |
| R | 6.8 | 6.3 | 5.6 | 6.1 | 1.36 | 1.26 | 1.12 | 2.44 | 6.18 |
| S | 7.3 | 5.7 | 6 | 5 | 1.46 | 1.14 | 1.2 | 2 | 5.8 |
| T | 6.8 | 6.2 | 7.2 | 6.3 | 1.36 | 1.24 | 1.44 | 2.52 | 6.56 |

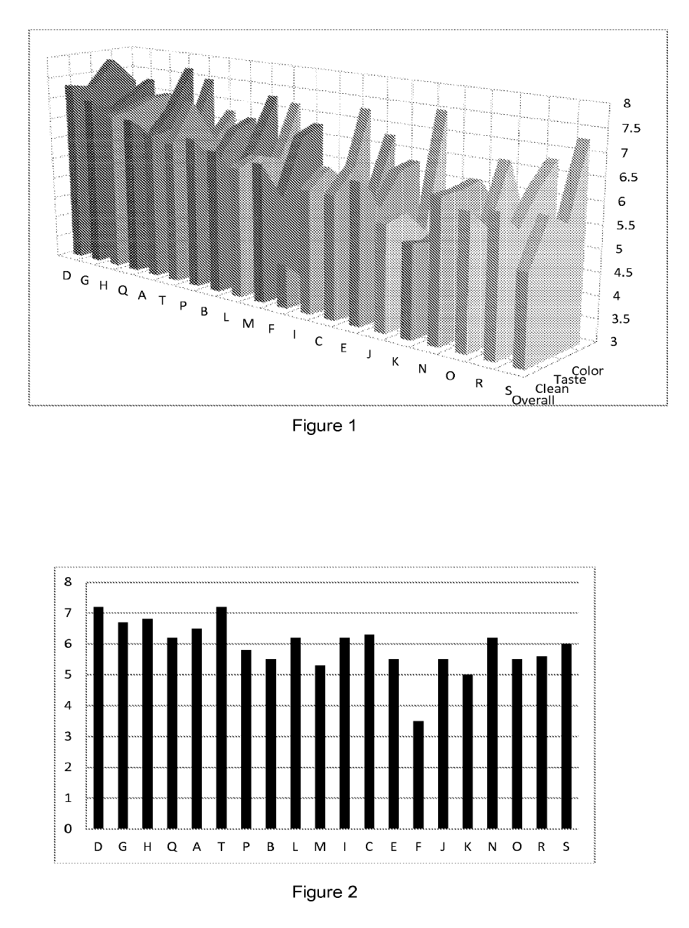

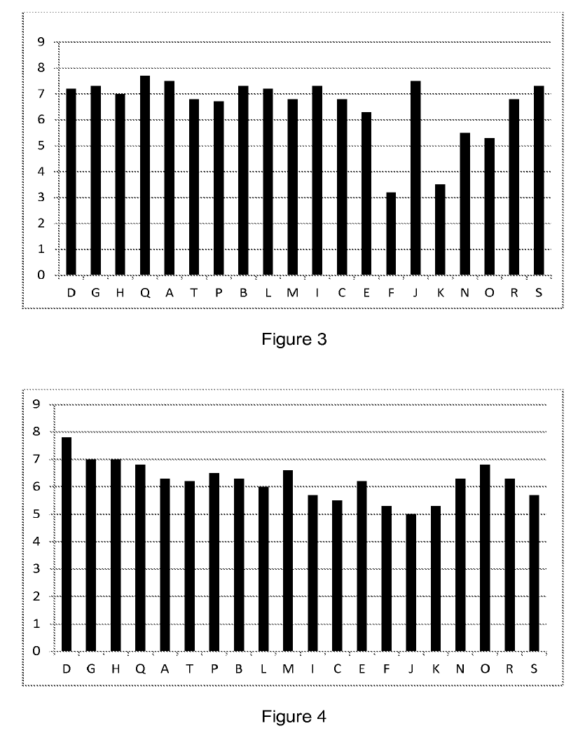

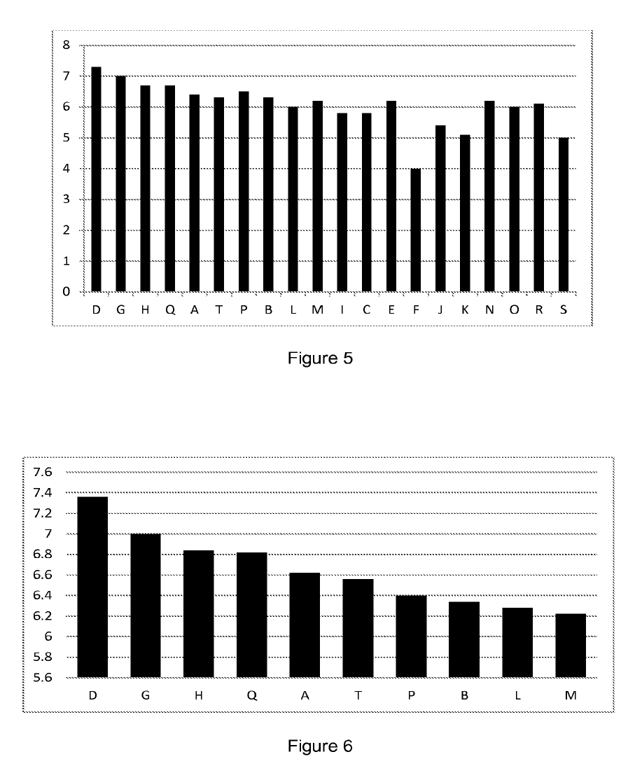

Graphical representations of some results of Table 3 are provided in FIGS. 1-6. FIGS. 1, 2, 3, 4, 5, 6 show graphic assessments of some embodiments of the invention, respectively for the cleansing-, color- and taste-sensory experience, and overall cleansing effectiveness. It is important to note that the taste-related experience seems to be more significant for the cleansing sensory experience than the color-related one. It is very well demonstrated by the results of composition D, for instance. This highlights the importance of properly choosing the soothing agents alongside the cleansing agents and, thus, the importance of the concept underlying this invention.

The set of results in FIG. 6 demonstrates several different combinations of cleansing and soothing agents which presented an above-average overall cleansing effect. These data corroborate that the unique combination of cleansing and/or soothing agents of the invention provides an improved sensory experience. Those skilled in the art will readily appreciate the teachings herein provided, and will be enabled to reproduce the invention according to the examples shown above or in other embodiments, which shall be deemed as within the scope of the invention and of the appended claims.

1. Personal care cleansing composition promoting improved and long-lasting cleansing sensory experience comprising a combination of multi-charged, negatively-charged, positively-charged and uncharged cleansing and/or soothing agents.

2. Composition according to claim 1 wherein said combination comprises at least seven cleansing and/or soothing agents.

3. Composition according to claim 2 wherein said combination comprises at least four cleansing agents and at least three soothing agents.

4. Composition according to claim 3 characterized by being alcohol-free and comprising a flavor-producing soothing agent or a combination of flavor-producing soothing agents.

5. Composition according to claim 4 comprising agents selected from mild cleansing agents, non-ionic surfactant cleansers, quaternary surfactant cleansers, multiquaternary surfactants, phospholipids, alkyl polyphosphates, alkyl betaines, alkyl hydroxysultaines, alkyl sulfosuccinates, polyphosphates, antiseptics, mild amino acid surfactant cleansers, cooling agents, soothing mint flavor agents, soothing tingling sensate, sweet soothing agents, soothing botanical agents and soothing flavor agents, or combinations thereof.

6. Composition according to claim 5 comprising: from 0.01 to 10% w/w of at least one uncharged cleansing agent, from 0.01 to 10% w/w of at least one positively-charged cleansing agent, from 0.001 to 10% w/w of at least one multi-charged cleansing agent, from 0.001 to 10% w/w of at least one negatively-charged cleansing agent, from 0.001 to 10% w/w of at least one soothing tingling sensate agent, from 0.01 to 0.5% w/w of at least one soothing mint flavor agent, from 0.01 to 1% w/w of at least one sweet soothing agent, from 0.001 to 1% w/w of at least one soothing botanical agent, from 0.001 to 5% w/w of at least one soothing flavor agent.

7. Composition according to claim 6 wherein said cleansing and/or soothing agents are selected from poloxamer 407, chlorhexidine, octenidine, lecithin, cephalin, sphingomyelin, phosphatidylserine, phosphatidic acid, phosphatidylinositol, phosphatidylinositol phosphate, phosphatidylinositol diphosphate, phosphatidylinositol triphosphate, nucleosidic polyphosphates (adenosine diphosphate, adenosine triphosphate, uridine triphosphate, thymidine triphosphate, cytidine triphosphate, guanosine triphosphate), glycosyl polyphosphates (inositol polyphosphate, glucose polyphosphate), sodium hydroxypropylphosphate laurylglucoside crosspolymer, phosphatidylinositol diphosphate, phosphatidylinositol triphosphate, cocamidopropyl betaine, behenyl betaine, cetyl betaine, decyl betaine, oleyl betaine, stearyl betaine, lauryl betaine, myristyl betaine, tallow betaine, cocamidopropyl hydroxysultaine, lauryl hydroxysultaine, erucamidopropyl hydroxysultaine, lauramido hydroxysultaine, oleamidopropyl hydroxysultaine, tallowamidopropyl hydroxyl sultaine, dioctyl sodium sulfosuccinate, disodium laureth sulfosuccinate, disodium lauryl sulfosuccinate, sodium hexametaphosphate, Sodium trimetaphosphate, cetylpyridinium chloride, polysorbate 20, sodium lauroyl sarcosinate, tetrasodium pyrophosphate, benzyl alcohol, Chattem sensate blend 709053, sugar free sweetener, Aloe vera, methyl salicylate, menthol and Chattem sensate blend 112683.

8. Composition according to claim 7 further comprising 0.01 to 0.5% of sodium fluoride.

9. Composition according to claim 8 comprising:
from 0.01 to 0.5% w/w of sodium fluoride;
from 0.5 to 5% w/w of poloxamer 407;
from 0.01 to 1% w/w of cetylpyridinium chloride;
from 0.01 to 10% w/w of polysorbate 20;
from 0.001 to 10% w/w of sodium lauroyl sarcosinate;
from 0.001 to 1% w/w of tetrasodium pyrophosphate;
from 0.1 to 5% w/w of benzyl alcohol;
from 0.01 to 0.5% w/w of Chattem sensate blend 709053;
from 0.01 to 1% w/w of sugar free sweetener;
from 0.001 to 1% w/w of Aloe vera;
from 0.001 to 5% w/w of methyl salicylate;
from 0.01 to 2% w/w of menthol;
from 0.001 to 10% w/w of Chattem sensate blend 112683; and
from 0.1 to 99% w/w of water.

10. Pre-mix for preparing liquid, semi-solid or solid personal care cleansing compositions characterized by comprising a combination of multi-charged, negatively-charged, positively-charged and uncharged cleansing and/or soothing agents.

11. Pre-mix according to claim 10 wherein said combination comprises at least seven cleansing and/or soothing agents.

12. Pre-mix according to claim 11 wherein said combination comprises at least four cleansing agents and at least three soothing agents.

13. Pre-mix according to claim 12 characterized by being alcohol-free and comprising a flavor-producing soothing agent or a combination of flavor-producing soothing agents.

14. Process for preparing personal care cleansing compositions comprising the following steps:
adding to a container, individually or pre-mixed, multi-charged, negatively-charged, positively-charged and uncharged cleansing and/or soothing agents; and
completing the volume with the acceptable carrier for liquid, semi-solid or solid final form.

15. Process according to claim 14 further comprising the introduction, either individually or pre-mixed, of other ingredients selected from: moisturizer, solvent, humectant, sweetener, preservative, buffer, metal chelator, color additive and combinations thereof.

16. Process according to claim 15 comprising the following steps:
adding, individually or pre-mixed to a container comprising water, at least one uncharged cleansing agents, at least one positively-charged cleansing agent, at least one multi-charged cleansing agent, at least one negatively-charged cleansing agent and then homogenizing the resulting mixture;
adding, individually or pre-mixed to a container comprising the previously formed mixture, at least one soothing tingling sensate agent, at least one soothing mint flavor agent, at least one soothing botanical agent and at least one soothing flavor agent; and
completing the volume with deionized, sterile or purified water

17. Process according to claim 16 comprising the following steps:
adding, individually or pre-mixed to a container comprising water, from 0.01 to 10% w/w of at least one uncharged cleansing agent, from 0.01 to 10% w/w of at least one positively-charged cleansing agent, from 0.001 to 10% w/w of at least one multi-charged cleansing agent, from 0.001 to 10% w/w of at least one negatively-charged cleansing agent, and then homogenizing the resulting mixture;
adding, individually or pre-mixed to a container comprising the previously formed mixture, from 0.001 to 10% w/w of at least one soothing tingling sensate agent, from 0.01 to 0.5% w/w of at least one soothing mint flavor agent, from 0.01 to 1% w/w of at least one sweet soothing agent, from 0.001 to 1% w/w of at least one soothing botanical agent, from 0.001 to 5% w/w of at least one soothing flavor agent; and
completing the volume with deionized, sterile or purified water.
